(19) United States
(12) Patent Application Publication
(10) Pub. No.: US 2008/0114416 A1
(43) Pub. Date: May 15, 2008

(54) STIMULATION PROGRAMMER WITH CLINICALLY-ADAPTIVE MODALITY

(75) Inventors: Margaret E. Theriot, Willis, TX (US); Kerry Bradley, Glendale, CA (US); Holly A. Segel, Englewood, CO (US)

Correspondence Address: Vista IP Law Group LLP, 2040 MAIN STREET, 9TH FLOOR, IRVINE, CA, 92614, US

(73) Assignee: ADVANCED BIONICS CORPORATION, Valencia, CA (US)

(21) Appl. No.: 11/559,377

(22) Filed: Nov. 13, 2006

(51) Int. Cl. A61N 1/08

(52) U.S. Cl. 607/59

(57) ABSTRACT

Tissue stimulation systems generally include a pulse generating device for generating electrical stimulation pulses, at least one implanted electrode for delivering the electrical stimulation pulses generated by the pulse generating device, and a programmer capable of communicating with the pulse generating device. In tissue stimulation systems, a clinically-adaptive stimulation programmer may be utilized, wherein a user communicates to the programmer a purpose of a programming session and a person who is to control the programming session. The clinically-adaptive stimulation programmer may be capable of determining a series of steps required to implement the programming session based on the selected purpose and the selected person. The clinically-adaptive programmer may implement the determined series of steps and communicate with the selected person during the programming session. Also provided are programming methods employing the clinically-adaptive programmer.

### BACKGROUND OF THE INVENTION

The present invention relates to tissue stimulation systems and more particularly to a clinically-adaptive stimulation programmer.

One example of a stimulation system is a spinal cord stimulation (“SCS”) system. Spinal cord stimulation is a well-accepted clinical method for reducing pain in certain populations of patients. An SCS system typically includes an Implantable Pulse Generator (IPG) or a radio-frequency (RF) transmitter and receiver, electrodes, electrode leads, and when necessary, lead extensions. The electrodes are implanted along the dura of the spinal cord, and the IPG or RF transmitter generates electrical pulses that are delivered, through the electrodes, to the dorsal column and dorsal root fibers within the spinal cord. Individual electrode contacts (the “electrodes”) are arranged in a desired pattern and spacing in order to create an electrode array. Individual wires within one or more electrode leads connect with each electrode in the array. The electrode leads exit the spinal column and attach to one or more electrode lead extensions, when necessary. The electrode leads or extensions are typically tunneled along the torso of the patient to a subcutaneous pocket where the IPG or RF-receiver is implanted.

Spinal cord stimulators and other stimulation systems are known in the art. For example, an implantable electronic stimulator is disclosed in U.S. Pat. No. 3,646,940 issued Mar. 7, 1972 for “Implantable Electronic Stimulator Electrode and Method” that provides timed sequenced electrical impulses to a plurality of electrodes. As another example, U.S. Pat. No. 3,724,467 issued Apr. 3, 1973 for “Electrode Implant For The Neuro-Stimulation of the Spinal Cord,” teaches an electrode implant for the neuro-stimulation of the spinal cord. A relatively thin and flexible strip of physiologically inert plastic is provided as a carrier on which a plurality of electrodes is formed. The electrodes are connected by leads to an RF receiver, which is also implanted.

In U.S. Pat. No. 3,822,708, issued Jul. 9, 1974 for “Electrical Spinal Cord Stimulating Device and Method for Management of Pain,” another type of electrical spinal cord stimulation device is taught. The device disclosed in the '708 patent has five aligned electrodes, which are positioned longitudinally on the spinal cord. Electrical pulses applied to the electrodes block perceived intractable pain, while allowing passage of other sensations. A patient operated switch allows the patient to adjust the stimulation parameters.

An SCS system treats chronic pain by providing electrical stimulation pulses through the electrodes of an electrode array located at the distal end of a lead placed epidurally next to a patient's spinal cord. The combination of electrodes used to deliver stimulation pulses to the targeted tissue constitutes an electrode configuration. In other words, an electrode configuration represents the polarity, being positive, negative, or zero, and for certain SCS systems with such capabilities, relative percentage of the current provided through each of the electrodes. Electrode arrays used with known SCS systems may employ between 1 and 16 electrodes on a lead. Electrodes are selectively programmed to act as anodes, cathodes, or left off, creating an electrode configuration. The number of electrodes available, combined with the ability to generate a variety of complex stimulation pulses, presents a huge selection of electrode configurations and stimulation parameters (together referred to herein as “stimulation sets”) to the clinician.

Other parameters that may be controlled or varied in SCS are the frequency of pulses provided through the electrode array, pulse width, and the amplitude of pulses delivered. Amplitude may be measured in milliamps, volts, etc., as appropriate, depending on whether the system provides stimulation from current sources or voltage sources. With some SCS systems, the distribution of the current/voltage across the electrodes (including the case of the pulse generator or receiver, which may act as an electrode) may be varied such that the current is supplied via numerous different electrode configurations. In different configurations, different combinations of electrodes may provide current (or voltage) in different relative percentages of positive and negative current (or voltage). Moreover, there may be some electrodes that remain inactive for certain electrode configurations, meaning that no current is applied through the inactive electrode.

Programming processes are described in U.S. Pat. No. 6,622,048, herein incorporated by reference in its entirety. A stimulation programmer is utilized to instruct the pulse generating device to generate electrical stimulation pulses in accordance with selected parameters or stimulation sets. A stimulation programmer may be programmed by a technician attending the patient. A stimulation programmer may be used in several scenarios. For example, when an SCS system is implanted, a procedure is performed to assure that the leads and/or electrodes are properly implanted in effective locations in the body. Such a session of applying electrical stimulation to test placement of the leads and/or electrodes may be referred to as an operating room (OR) mapping procedure. A navigation session is a fitting procedure to select one or more effective stimulation sets for a particular patient. Such a session generally occurs after the leads and/or electrodes are implanted into a patient. Other programming sessions may include an extensive fitting procedure, a follow-up procedure, and an addition of a program procedure.

One known programmer for an IPG for spinal cord stimulation is called the Bionic Navigator™, available from Advanced Bionics Corp., Sylmar, Calif. The Bionic Navigator™ employs current-steering algorithms to electrically “steer” the supplied current along the implanted leads in real-time. The Bionic Navigator™ is a software package that operates on a suitable PC and allows clinicians to program all stimulation parameters on each channel, including the current distribution to the contacts (as % cathodic current, % anodic current or off).

The Bionic Navigator™ generally displays four (4) screens to the attending clinician for programming. These screens include a threshold calibration screen, a navigator screen, an area screen, and a remote screen. These programming screens are used to set patient thresholds for the stimulation therapy and to initialize IPG for a therapy session. Programming through these screens serves to find suitable parameters to use during the stimulation therapy.

Programming sessions with a stimulation programmer may be complex and time-consuming. A clinician generally has to perform 100% of the programming of the patient's stimulator. Programming may involve setting thresholds and parameters, as well as testing several stimulation sets. As explained in reference to the Bionic Navigator™, generally, a number of display screens are viewed by a clinician who inputs the requested information with patient involvement. This process can be limiting due to the time and complexity involved.

Thus, there is a need to reduce the burden on the clinician of tedious and time-consuming programming. There is a need to develop an adaptive stimulation programmer which may lead a user through a series of programming steps. By having an adaptive stimulation programmer, the clinician would be less burdened and more users would be encouraged to perform the programming.

### SUMMARY OF THE INVENTION

The present invention addresses the above and other needs by providing a clinically-adaptive stimulation programmer and methods for programming the adaptive programmer.

In accordance with a first aspect of the present invention, a method for programming a tissue stimulation system having a pulse generating device and at least one implantable electrode for delivering electrical stimulation pulses, is provided. The method comprises selecting one or more of a plurality of purposes and/or one or more of a plurality of user types for a programming session. The purpose(s) may, e.g., be selected from the group consisting of mapping, fitting, extensive fitting, follow-up modification, and addition of program, and the user type(s) may, e.g., be selected from the group consisting of a patient, technician, and clinician.

The method further comprises automatically determining a series of steps required to implement the programming session based on the selected purpose(s) and/or user type(s), and performing the determined series of steps using the electronic programmer to program the pulse generating device. In one method, the series of steps can be automatically determined by the electronic programmer. In an optional method, the series of steps comprises displaying one or more programming screens. In this case, the method may further comprise inputting one or more programming choices through the screens, and communicating the choices from the electronic programmer to the pulse generating device.

In accordance with a second aspect of the present inventions, a tissue stimulation system is provided. The tissue stimulation system comprise a pulse generating device for generating electrical stimulation pulses, at least one implantable electrode for delivering the electrical stimulation pulses, and an electronic programmer configured for receiving a selection of one or more of a plurality of purposes for a programming session and/or for receiving a selection of one or more of a plurality of user types, determining a series of steps required to implement the programming session based on the selected one or more purposes and/or selected one or more user types, and performing the determined series of steps to program the pulse generating device. The tissue stimulation system may optionally comprise an interface device configured for communicating the selected purpose(s) and/or user type(s) to the electronic programmer. In this case, the interface device may comprise a display device capable of displaying one or more programming screens to the selected user, and receiving from the selected user one or more programming choices through the screens. The electronic programmer may communicate these choices to the pulse generating device.

### BRIEF DESCRIPTION OF THE DRAWINGS

The above and other aspects of the present invention will be more apparent from the following more particular description thereof, presented in conjunction with the following drawings wherein:

FIG. 1 depicts a Spinal Cord Stimulation (SCS) system, as an example of a tissue stimulation system.

FIG. 2 depicts the SCS system of FIG. 1 implanted in a spinal column.

FIG. 3 depicts a process of programming a pulse generator.

FIG. 4 depicts a user interface display that may be used during a programming session.

FIG. 5 depicts a user interface device that may be used during a programming session.

### DETAILED DESCRIPTION OF THE EMBODIMENTS

It is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to an “electrode” is a reference to one or more electrodes and equivalents thereof known to those skilled in the art, and so forth. Unless defined otherwise, all technical and scientific terms used herein have the same meanings as commonly understood by one of ordinary skill in the art. Although any methods, devices, and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

The methods of the present invention provide programming methods of a stimulation programmer used in connection with a tissue stimulation system. A Spinal Cord Stimulation (SCS) system will be used herein as an example of such a tissue stimulation system.

The various components of an exemplary SCS system may include an implantable pulse generator (IPG) and programmer used with such system. Implantable components may include an implantable pulse generator, one or more electrode arrays, and (as needed) one or more extensions to connect the array(s) to the IPG. Such implantable components, external devices and circuitry are more fully described in U.S. Pat. No. 6,622,048. Alternatively, a system comprised of an implanted RF receiver and external transmitter, as a pulse generating device in place of an IPG, may be used.

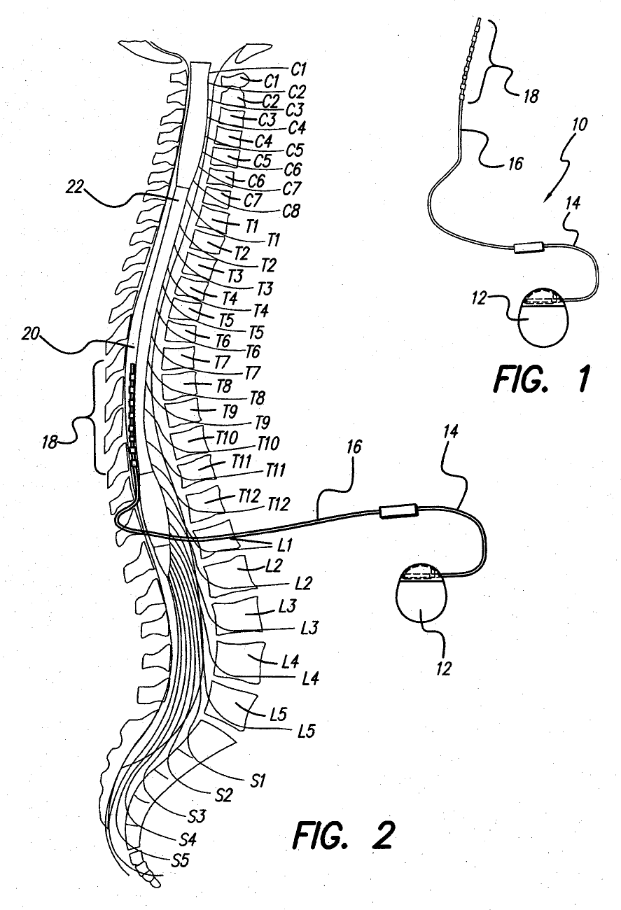

An exemplary Spinal Cord Stimulation (SCS) system 10 is shown in FIG. 1. SCS system 10 comprises an Implantable Pulse Generator (IPG) 12, an optional lead extension 14, an electrode lead 16, and an electrode array 18. The IPG 12 generates stimulation current for implanted electrodes that make up the electrode array 18. When needed, a proximal end of the lead extension 14 is removably connected to the IPG 12 and a distal end of the lead extension 14 is removably connected to a proximal end of the electrode lead 16. Alternatively, a proximal end of lead 16 is attached directly to the IPG 12. Electrode array 18 is formed on a distal end of the electrode lead 16. The in-series combination of the lead extension 14 and electrode lead 16, carry the stimulation current from the IPG 12 to the electrode array 18.

The SCS system 10 described in FIG. 1 above is depicted implanted in the epidural space 20 in FIG. 2. The electrode array 18 is implanted at the site of nerve fibers that are the target of stimulation, e.g., along the spinal cord. Due to the lack of space near the location where the electrode lead 16 exits the spinal column, the IPG 12 is generally implanted in the abdomen or above the buttocks. When needed, the lead extension 14 facilitates locating the IPG 12 away from the electrode lead exit point. Another example of a SCS system that may be used with the present invention is described in U.S. Pat. No. 6,516,227, incorporated herein by reference in its entirety. Another stimulation system is described in U.S. Pat. No. 6,393,325 and related applications and issued patents. It is to be emphasized, however, that the invention herein described may be used with many different types of stimulation systems, and is not limited to use with the representative SCS system.

As explained in the background section, a stimulation programmer is utilized to instruct the pulse generating device to generate electrical stimulation pulses in accordance with selected parameters or stimulation sets. A stimulation programmer may be programmed by a technician attending the patient. A stimulation programmer may be used in several scenarios. For example, when an SCS system is implanted, an operating room (OR) mapping procedure is performed to assure that the leads and/or electrodes are properly implanted in effective locations in the body. Additionally, a navigation session is a fitting procedure to select one or more effective stimulation sets (which typically include specific electrode configuration and stimulation amplitudes) for a particular patient. Such a session generally occurs after the leads and/or electrodes are implanted into a patient. Other programming sessions may include an extensive fitting procedure, a follow-up procedure, and an addition of a program procedure.

An extensive fitting procedure can occur at any time, and may be performed to identify stimulation parameters to treat one or more areas of pain with one or more sets of stimulation parameters. It usually occurs when it is determined that the sufficient stimulation sets cannot be determined during the navigation session, in which case, a whole host of stimulation parameters, including electrode arrangement, amplitude value, pulse width value, and pulse rate, can be modified, typically by a technician or clinician. A follow-up procedure is a fine-tuning procedure wherein a patient has previously had a fitting procedure and is simply making minor adjustments to stimulation parameters. An additional program, corresponding to a different pain, may be added to the patient's pain management therapy. Different programs may be used to treat different patient states. For example, back pain when lying down vs. back pain while sitting may require different stimulation parameters. Other programming scenarios are possible and are suitable for the present invention methods.

A stimulation programmer may interface with a user device and also with the implanted pulse generator. Programmers may be in the form of a conventional PC, a laptop, a PDA, a monitor, a hand-held device, and any other suitable computing means.

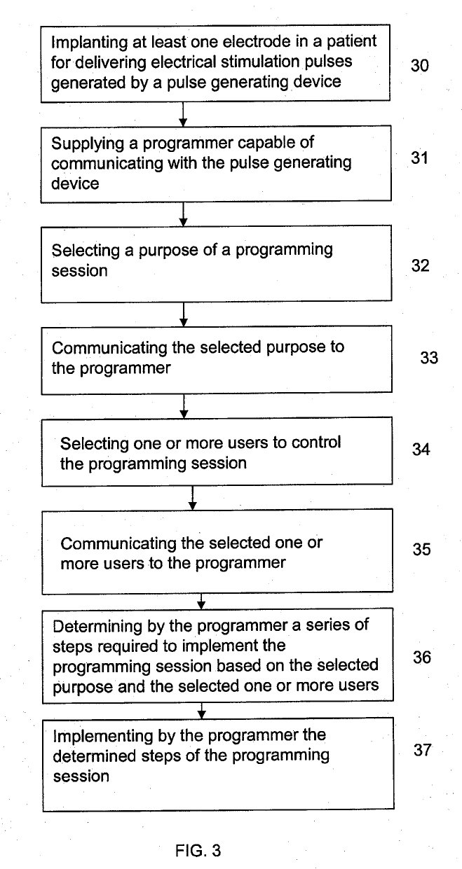

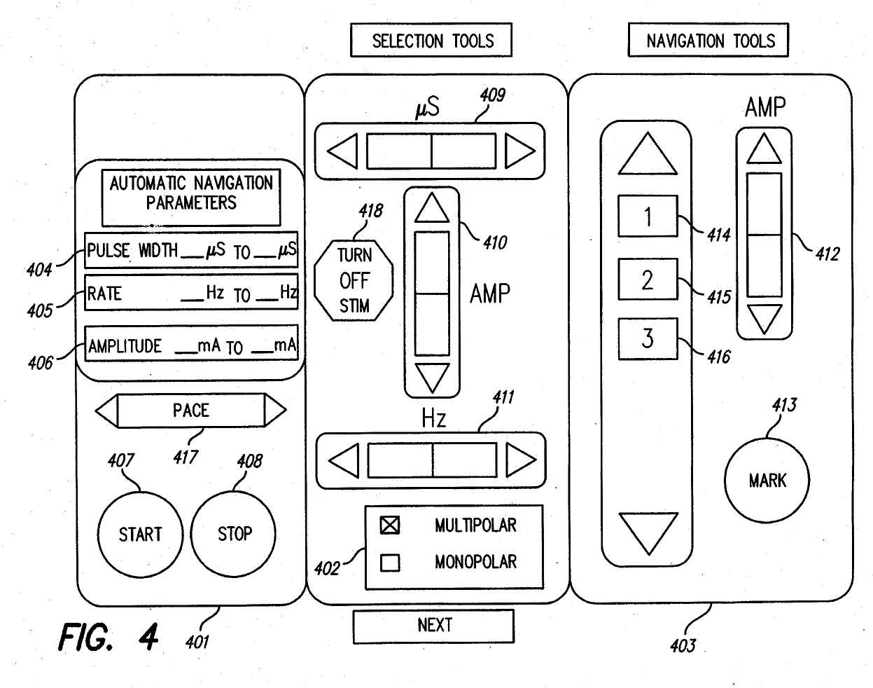

One method for programming a tissue stimulation system is illustrated in FIG. 3. At step 30, at least one electrode may be implanted in a patient for delivering electrical stimulation pulses generated by a pulse generating device. At step 31, a programmer capable of communicating with the pulse generating device may be supplied. A user may then select a purpose of a programming session, at step 32. The purpose may be any of the scenarios discussed above, such as a fitting procedure, an extensive fitting procedure, a mapping procedure, a navigation procedure, a follow-up procedure, and an addition of a program procedure. The selected purpose may be communicated to the stimulation programmer, at step 33. A user may specify one or more user types to control the programming session, at step 34. These selected user(s) may be communicated to the stimulation programmer, at step 35.

With the entered information, the stimulation programmer determines a series of steps required to implement the programming session, at step 36. In step 37, after the programmer determines the series of steps, the programmer implements the determined steps of the programming session. These steps are communicated to the user, who may be prompted to enter specific information. For example, the user may be requested to enter information regarding a series of stimulation parameters that are being tested on the patient during a programming session. There are several methods for leading a user through a series of stimulation parameters in order to identify effective sets of parameters for a stimulation session. Any of these methods may be used in connection with the present programming methods.

For example, in U.S. Pat. No. 6,393,325, herein incorporated by reference in its entirety, a system of testing a series of stimulation parameters in a systematic method is described, wherein a patient may direct the movement of the stimulus current through a suitable interface. In U.S. Pat. No. 6,622,048, a method of using a pain map or a pictorial illustration of the human body and anatomical relationships between the spine and body is disclosed in connection with programming methods. Other methods of testing the effectiveness of various stimulation parameters are disclosed in U.S. application Ser. Nos. 11/026,859 and 11/105,643, each herein incorporated by reference in their entirety. These methods include using parameter tables during a fitting session to step through and optimize stimulation parameters. The present invention therefore, is not limited to a particular method of testing stimulation parameters, but instead describes a method of selecting appropriate instructions to be given to a user to guide her through the testing process.

In selecting the steps to implement the programming session, the programmer may make the determination through any suitable algorithm or programming logic. For instance, if a patient is the user and the intent of the programming session is a follow-up procedure, a minimum number of simplified steps may be presented to the patient. The programmer may be equipped with a database of suitable programming steps. With the entered information of user and intent, the programmer is able to select an appropriately stored programming series of steps from its database and present these steps to the user. The programmer may be able to control the speed at which programming steps are displayed to a patient-user.

As another example, if the device technician is selected as the user type, the programmer realizes that the technician is skilled at programming and may present, relative to the number of instructions that would be presented to a patient, an increased number of instructions to the technician for programming.

The series of steps is carried out on the programmer, such as through displaying a number of programming screens to the selected user. The screens may be displayed through any suitable interface device. Interfaces may include, but are not limited to, display screens, handheld devices, monitors, laptops, and PDAs. The interfaces may be interactive, such as a touch screen. The user may use a mouse, joystick, or stylus in connection with the interface for the inputting her selections during programming. Thus, the selected user may input the programming choices through the display screens. The programmer may receive and store the choices and thus use this input to determine the nature of the electrical stimulation to be applied to the patient. The programmer communicates the choices or user input to the pulse generating device. In one embodiment, the user may select one or more stimulation sets during the programming session. The stimulation sets may be stored by the programmer and communicated to the pulse generating device to generate electrical stimulation pulses in accordance with the stimulation set.

The user types may be selected from the group consisting of patient, technician, clinician, and combinations thereof. In one embodiment, the patient may be the selected user in order to allow maximum patient control. In another embodiment, the patient and attending clinician may share control of the programming session. In another embodiment, the clinician may specify the level of patient control. Because no two patients are alike, the degree of patient control may be assessed for each patient. Thus, a stimulation programmer that allows the clinician to select the degree, level or amount of patient control would be more time efficient and allow for individualized programming sessions. Allowing an appropriate level of patient control reduces patient anxiety over the programming session and also enhances the effectiveness of patient/clinician communication.

Users may use a handheld device or other suitable interface that allows communication with the programmer. The interface allows the user to respond to the programmer's requests for input, such as the adjusting of threshold stimulation parameters. Any suitable user interface may be incorporated into embodiments of the invention. For example, the interfaces described in U.S. Pat. No. 6,393,325 may be used or altered for the programming sessions described herein.

As another example, the interface displayed in FIG. 4 may be used to guide a user through the programming session. As seen in FIG. 4, the interface may include three panels, or any combination or portion of the three panels (401, 402, 403). The user may be prompted to enter the parameters displayed in the 401 panel. These parameters may be set such as pulse width 404, rate 405 and amplitude or strength 406. The interface may also have a start 407 and stop 408 switch that halts or resumes the programming, respectively. The user may be able to adjust the pulse width 409, amplitude 410 or rate 411, as well as entirely halt delivery of stimulation pulses, i.e., turn simulation off 418, within the interface displayed at panel 402. In panel 403, a user may be able to adjust the amplitude 412. The user is also able to highlight, mark, or select 413 the electrode configurations being tested. The user may be able to select from 414, 415, and 416, which correspond to sets of electrode configurations to be tested. Finally, the pace 417 may be varied during the navigation so as to adjust the speed at which consecutive electrode configurations are applied.

The user is prompted by the steps of the programming session to use this display screen to enter the requested information regarding the parameters. In other words, the screen displayed in FIG. 4 may be used in combination with other programming instructions. For example, another screen or a voice-over may instruct the user using the screen displayed in FIG. 4. As another example, various portions of the screen illustrated in FIG. 4 may be highlighted or displayed to the user in a suitable order, to guide the user to enter the requested information.

Although the interface controls of FIG. 4 are illustrated as being a touch screen, any other interface device that allows adjustment of these various parameters may be designed. For example, a hand-held user control device may be used having these parameter controls. Also, although the controls of FIG. 4 may appear to be “buttons” any other suitable controls may be used, such as sliding scales or dials.

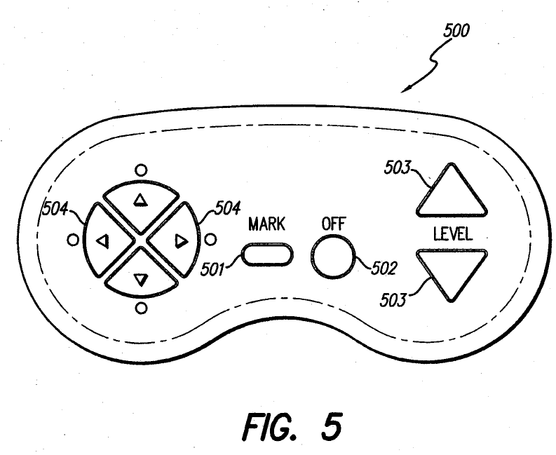

Another user device for allowing user adjustment of the stimulation parameters is depicted in FIG. 5. The hand-held device 500 may be small and easy to manipulate. The patient is given control to mark, highlight or select 501 electrode configurations. Additionally, the patient may turn the navigation session “off” 502 with a suitable safety or escape button. The patient may adjust the amplitude 503 through a pair of increase and decrease buttons. Finally, with a series of four directional buttons 504, the patient may be able to gradually shift paresthesia locations on the body until pain coverage is obtained.

As explained in reference to FIG. 4, the user is prompted by the steps of the programming session in how to use the device of FIG. 5. For example, the device of FIG. 5 may be used in connection with another display screen prompting the user to select various stimulation parameters.

In programming sessions, control may be parallel between the clinician and the patient. However, based upon the patient's level of control, the patient may be given priority of control over a clinician, effectively allowing the patient control to override the clinician control. Such priority to the patient's selection, decisions, and control may be given only to specific parameters. For example, the patient may be given priority control for the adjustment of stimulation amplitude (strength). In one embodiment, the clinician uses the interface described in FIG. 4, while the patient uses the hand-held device depicted in FIG. 5. The selection of a suitable hand-held device may depend on patient sophistication. In other words, a patient may “graduate” from a simplified device to a more advanced device, allowing her greater control over the programming session.

The methods of the present invention may be incorporated into any tissue stimulation system, such as any SCS, neural or muscle stimulation system. Thus, in another embodiment, a tissue stimulation system is provided. A system may comprise: (1) a pulse generating device for generating electrical stimulation pulses; (2) at least one implanted electrode for delivering the electrical stimulation pulses generated by the pulse generating device; (3) a programmer, wherein the programmer is capable of instructing the pulse generating device to generate electrical stimulation pulses; and (4) an interface device for communicating with the programmer. A user may communicate to the stimulation programmer through the interface device a purpose of a programming session and a person who is to control the programming session. With this information, the stimulation programmer may be capable of determining a series of steps required to implement the programming. The stimulation programmer may be capable of implementing the determined steps and communicating with the selected person during the programming session.

While the invention herein disclosed has been described by means of specific embodiments and applications thereof, numerous modifications and variations could be made thereto by those skilled in the art without departing from the scope of the invention set forth in the claims. For example, the methods discussed above are not limited to spinal cord stimulation systems and may be used with many kinds of stimulation systems such as, but not limited to, those described above, cochlear implants, cardiac stimulation systems, peripheral nerve stimulation systems, muscle tissue stimulation systems, brain stimulation systems and microstimulators.

What is claimed is:

1. A method for programming a tissue stimulation system having a pulse generating device and at least one implantable electrode for delivering electrical stimulation pulses, the method comprising:
selecting one or more of a plurality of purposes for a programming session;
automatically determining a series of steps required to implement the programming session based on the selected one or more purposes; and
performing the determined series of steps using the electronic programmer to program the pulse generating device.

2. The method of claim 1, wherein the at least one purpose is selected from the group consisting of mapping, fitting, extensive fitting, follow-up modification, and addition of program.

3. The method of claim 1, wherein the determined series of steps comprises displaying one or more programming screens.

4. The method of claim 3, further comprising inputting one or more programming choices through the screens, and communicating the choices from the electronic programmer to the pulse generating device.

5. The method of claim 1, wherein the series of steps are automatically determined by the electronic programmer.

6. The method of claim 1, further comprising selecting at least one of a plurality of user types, wherein the series of steps required to implement the programming session are automatically determined further based on the one or more selected user types.

7. A method for programming a tissue stimulation system having a pulse generating device and at least one implantable electrode for delivering electrical stimulation pulses, the method comprising:
selecting one or more of a plurality of user types for a programming session;
automatically determining a series of steps required to implement the programming session based on the selected one or more user types; and
performing the determined series of steps using the electronic programmer to program the pulse generating device.

8. The method of claim 7, wherein the at least one user type is selected from the group consisting of a patient, technician, and clinician.

9. The method of claim 7, wherein the determined series of steps comprises displaying one or more programming screens.

10. The method of claim 9, further comprising inputting one or more programming choices through the screens, and communicating the choices from the programmer to the pulse generating device.

11. The method of claim 8, wherein the series of steps are automatically determined by the electronic programmer.

12. The method of claim 8, wherein the determination of the series of steps comprises minimizing a number of the steps based on the selected one or more user types.

13. The method of claim 12, wherein the number of steps is minimized if the selected one or more user types is a patient.

14. A tissue stimulation system, comprising:
a pulse generating device for generating electrical stimulation pulses;
at least one implantable electrode for delivering the electrical stimulation pulses;
an electronic programmer configured for receiving a selection of one or more of a plurality of purposes for a programming session and/or for receiving a selection of one or more of a plurality of user types, determining a series of steps required to implement the programming session based on the selected one or more purposes and/or selected one or more user types; and performing the determined series of steps to program the pulse generating device.

15. The tissue stimulation system of claim 14, wherein the electronic programmer is configured for determining the series of steps based on the selected one or more purposes.

16. The tissue stimulation system of claim 14, wherein the electronic programmer is configured for determining the series of steps based on the selected one or more user types.

17. The tissue stimulation system of claim 14, wherein the electronic programmer is configured for determining the series of steps based on both the selected one or more purposes and the one or more user types.

18. The tissue stimulation system of claim 14, further comprising an interface device configured for communicating the selected one or more purposes and/or one or more user types to the electronic programmer.

19. The tissue stimulation system of claim 18, wherein the interface device comprises a display device capable of displaying one or more programming screens to the selected user.

20. The tissue stimulation system of claim 19, wherein the interface display device is capable of receiving from the selected user one or more programming choices through the screens, and the programmer is capable of communicating the choices to the pulse generating device.
